(19) United States
(12) Patent Application Publication
(10) Pub. No.: US 2025/0354179 A1
(43) Pub. Date: Nov. 20, 2025

(54) PROCESS FOR PRODUCING BIOGENIC CRYSTALS OF CALCIUM CARBONATE

(71) Applicant: UNIVERSITE DU LUXEMBOURG, Esch-sur-Alzette (LU)

(72) Inventors: Anupam SENGUPTA, Luxembourg (LU); Irvine Lian Hao ONG, Luxembourg (LU)

(21) Appl. No.: 19/106,882

(22) PCT Filed: Aug. 31, 2023
(86) PCT No.: PCT/EP2023/073889

§ 371 Date: Feb. 26, 2025

(30) Foreign Application Priority Data
Aug. 31, 2022 (LU) LU502733

(51) Int. Cl. C12P 3/00; C12N 1/20

(52) U.S. Cl. CPC C12P 3/00; C12N 1/20

(57) ABSTRACT

The present disclosure relates to a process for producing biogenic crystals of calcium carbonate, said process is remark-able in that it comprises the following steps: a) providing one or more colonies of CaCO3-producing bacteria; b) inoculating said one or more colonies in a first marine broth under inoculating conditions to form an inoculated culture; c) adding a second marine broth to the inoculated culture to form a dissolved inoculated culture and/or collecting a supernatant of the inoculated culture and adding a second marine broth to said supernatant; d) incubating said dissolved inoculated culture and/or said supernatant cell culture under incubating conditions to form a mixture of biogenic crystals of calcium carbonate, optionally, recovering said mixture of biogenic crystals of calcium carbonate.

### FIELD OF THE DISCLOSURE

The present disclosure relates to a process for producing biogenic crystals of calcium carbonate.

### BACKGROUND OF THE DISCLOSURE

Biogenic crystalline structures have been long observed in synthetic and natural ecosystems. Biogenic calcium carbonate has broad application prospects in biotechnology and civil engineering. Many of these structures, formed due to the interplay of microbial and environmental biochemistry, specifically biological exudates and the molecules present in the micro-environments, are biomineral precipitates, appearing as sub-millimetre to centimetre sized aggregates. Though mesoscale biogenic precipitation and aggregation show considerable potential in stabilizing subsurface foundations and under-water structures, including immobilization of hazardous contaminants in the subsurface (e.g., sequestration of CO₂ and storage of CH₄ or H₂), their application as microscale structures remain largely limited due to the lack of controllability of particle properties at micron or sub-micron scales. For instance, micron-sized particles find application in a variety of consumer goods and products of daily utility, including but not limited to hygiene and cosmetic products, paints, tyres, textiles, and even in food products. For decades, synthetic microplastics have been used as inclusions in the range of products we typically use on a daily basis, which—tragically—have plagued every nook and corner of our biosphere with microplastic pollution. As outlined by the recent IPCC and European Commission reports, microplastics pose an immediate and major environmental challenge, which if left unchecked could have detrimental and far-reaching ramifications on life across aquatic, terrestrial and atmospheric ecosystems.

In general, fast-moving consumer goods rely on state-of-the-art synthetic microplastic-based materials as fillers or inclusions. The application of biogenic particles as green alternatives is grossly unexplored.

In the context of paints, plastic-based microspheres and microfibers are used in building paints, rendering various properties, including crack scratch resistance, optimal elasticity and toughness. Though currently cellulose-based alternatives are being suggested, the production of cellulose itself involves collateral environmental impacts, as has been discussed in a recent European Commission report (see final report dated October 2017 and entitled “Intentionally added microplastics in products”). Additionally, tyre and road wares particles constitute a major source of environmental pollution (soil, fresh water and marine ecosystems), also in the form of microparticles which escape tyres due to wear and tear (see the scientific report on tyre and road wear particles (TRWP) in the aquatic environment by M. Jekel, dated Jul. 17, 2019). Yet, sustainable eco-friendly filler alternatives are largely missing. In the field of biologically-relevant or derived materials for various applications, which are aimed at replacing conventional materials based on non-renewable raw materials, several renewable processes are currently being explored and adopted. For example, plant-based materials are increasingly replacing conventional plastics used in consumable products. Another potential source of biologically-derived material is polyhydroxybutyrates (PHBs), which are typically produced by bacteria as inclusion bodies/particles under physiologically stressful conditions. However, the production costs of these materials have limited their wide production and usage.

US2011/0027850 makes use of the fact that many naturally occurring microorganisms present in geologically derived material, such as soil and/or rock, can hydrolyze urea in the presence of water to ammonium and carbonate ions. The increase of pH, due to the ammonium ions, and the presence of nucleation sites, favor the reaction between the carbonate ions and any calcium ions to form calcium carbonate. It was found that a source of nutrients can be added to the geologically derived material to promote the growth of microorganisms within the material.

The study by Rivadeneyra M. A., et al., entitled “Precipitation of calcium carbonate by Vibrio spp. from an inland saltern” (FEMS Microbiology Ecology, 1994, 13, 197-204) shows that the bacteria form magnesium calcite, with a variable Mg content, depending upon the medium provided. Even upon high Mg content, no aragonite was detected.

The study by Zhang C., et al., entitled “Controlled crystallization and transformation of carbonate minerals with dumbbell-like morphologies on bacterial cell templates” (Microscopy and Microanalysis, 2020, 1-12), has shown that amorphous particles nucleate on the surface of bacterial cell templates to form rod-like particles. Crystal growth follows at both ends of the rod-like particles, leading to dumbbell-like structures. The bacterial cell templates, from Curvibacter lanceolatus, were found to be necessary to form these structures. The dumbbell-like structures of calcite and/or aragonite in this study comprise magnesium and are formed following the ammonium carbonate diffusion method which requires the use of concentrated sulfuric acid to absorb the excess ammonia.

The study by Liu R., et al., entitled “Bio-mineralisation, characterization, and stability of calcium carbonate containing organic matter” (RSC Adv., 2021, 11, 14415) shows that Bacillus subtilis can induce various structural forms of CaCO₃, such as biogenic amorphous calcium carbonate (ACC) or biogenic vaterite. The fact that carbonic anhydrase, an enzyme capable of catalyzing the reversible hydration reaction (CO₂+H₂O⇄H⁺+HCO₃), secreted by the bacteria plays an important role in the mineralization of CaCO₃ was shown and it was demonstrated that upon addition of CaCl₂, the ACC was transformed to polycrystalline vaterite. It was also shown that the stability of ACC and vaterite is closely related to the protein and extracellular polysaccharide secreted by the bacteria. Thus, the protein may be inclined to inhibit the formation of calcite while the polysaccharide may be inclined to promote the formation of vaterite.

The present disclosure has for objective to find an alternative to the popular yet ecologically perilous microplastics by improving the processes of production of biogenic crystals of calcium carbonate such as calcite and/or aragonite.

### SUMMARY OF THE DISCLOSURE

According to a first aspect, the disclosure provides a process for producing biogenic crystals of calcium carbonate remarkable in that it comprises the following steps:
a) providing one or more colonies of CaCO₃-producing bacteria;
b) inoculating said one or more colonies in a first marine broth under inoculating conditions to form an inoculated culture;
c) adding a second marine broth to the inoculated culture to form a dissolved inoculated culture and/or collecting a supernatant of the inoculated culture and adding a second marine broth to said supernatant;
d) incubating said dissolved inoculated culture and/or said supernatant cell culture under incubating conditions to form a mixture of biogenic crystals of calcium carbonate,
e) optionally, recovering said mixture of biogenic crystals of calcium carbonate.

In a first embodiment, in the dissolved inoculated culture, the ratio between the inoculated culture and the second marine broth is ranging between 1/200 and 1/20; preferably, between 1/180 and 1/50; more preferably, between 1/150 and 1/100.

In a second embodiment, complementary or alternative to the first embodiment, the step of collecting a supernatant of step (c) is performed according to the following sub-steps:
i. ageing the inoculated culture to form an aged inoculated culture;
ii. centrifuging said aged inoculated culture to obtain a supernatant and a precipitate; and
iii. collecting the supernatant.

With preference, the ratio between the supernatant and the second marine broth is ranging between 1/3 and 3/1; preferably between 1/2 and 2/1.

With preference, said sub-step (i) is carried out during a period ranging between 2 days and 7 days, more preferably during a period ranging between 5 days and 7 days.

Surprisingly, it has been found that it is possible to provide a process to produce one or more biogenic crystals of calcium carbonate (CaCO₃) and in particular two of its polymorphs, namely calcite and/or aragonite, in an environmentally friendly manner using microbial structures and under smooth conditions, such as room temperature and/or aerobic conditions and/or without using any strong acid, allowing for selecting the final product since it is possible to adapt the composition of the marine broth that is used, in particular during step (c). In particular, the production of these calcium carbonate crystals is an example of the production of microbial biogenic tunable structures (μ-BITS) that have a wide spectrum of applications, such as consumer products, paints, and tyres. More particularly, the disclosure provides a process for producing biogenic crystals of calcium carbonate remarkable in that it comprises the following steps:
a) providing one or more colonies of CaCO₃-producing bacteria;
b) inoculating said one or more colonies in a first marine broth under inoculating conditions to form an inoculated culture;
c) adding a second marine broth to the inoculated culture to form a dissolved inoculated culture and/or ageing the inoculated culture to form an aged inoculated culture, centrifuging said aged inoculated culture to obtain a supernatant, collecting the supernatant, and adding a second marine broth to said supernatant to form a dissolved supernatant,
   wherein the second marine broth is selected to comprise less than 2.0 g/l of CaCl₂ or at least 2.5 g/l of CaCl₂;
d) incubating said dissolved inoculated culture and/or said dissolved supernatant under incubating conditions to form a mixture of biogenic crystals of calcium carbonate,
e) optionally, recovering said mixture of biogenic crystals of calcium carbonate.

This particular process for producing biogenic crystals of calcium carbonate allows to produce said crystals in a controlled manner. With preference, the biogenic crystals of calcium carbonate are aragonite and/or calcite.

For example, when the second marine broth is selected to comprise less than 2.0 g/l of CaCl₂, the second marine broth comprises 1.9 g/l of CaCl₂ or less, preferably 1.8 g/l of CaCl₂ or less, or 1.7 g/l of CaCl₂ or less, or 1.6 g/l of CaCl₂ or less, or 1.5 g/l of CaCl₂ or less. In that case, only aragonite is obtained.

For example, when the second marine broth is selected to comprise at least 2.5 g/l of CaCl₂, the second marine broth comprises between 2.6 g/l of CaCl₂ and less than 4.0 g/l of CaCl₂, or between 2.7 g/l of CaCl₂ and less than 4.0 g/l of CaCl₂, or between 2.8 g/l of CaCl₂ and 3.9 g/l of CaCl₂. In that case, biogenic crystals of calcium carbonate with a weight ratio of calcite over aragonite that is superior to 1 are obtained.

For example, when the second marine broth is selected to comprise at least 2.5 g/l of CaCl₂, the second marine broth comprises between at least 4.0 g/l of CaCl₂ and 6.5 g/l of CaCl₂ or more, or between 4.1 g/l of CaCl₂ and 6.4 g/l of CaCl₂, or between 4.2 g/l of CaCl₂ and 6.3 g/l of CaCl₂, or between 4.3 g/l of CaCl₂ and 6.2 g/l of CaCl₂, or between 4.2 g/l of CaCl₂ and 6.1 g/l of CaCl₂. In that case, only calcite is obtained.

With preference, in the dissolved inoculated culture, the ratio between the inoculated culture and the second marine broth is ranging between 1/200 and 1/20; preferably, between 1/180 and 1/50; more preferably, between 1/150 and 1/100.

With preference, in the dissolved supernatant, the ratio between the supernatant and the second marine broth is ranging between 1/3 and 3/1; preferably between 1/2 and 2/1. With preference, the step of ageing the inoculated culture to form an aged inoculated culture is carried out during a period ranging between 2 days and 7 days, more preferably during a period ranging between 5 days and 7 days.

With preference, step (a) comprises providing a single colony of CaCO₃-producing bacteria.

With preference, step (a) is performed by achieving the following sub-steps:
i. providing a culture medium with living CaCO₃-producing bacteria, said culture medium being a marine broth;
ii. adding glycerol in said culture medium;
iii. freezing said culture medium to obtain a frozen stock;
iv. streaking said frozen stock on an agar plate;
V. incubating the agar plate for at least 12 hours;
vi. extracting one or more colonies from said agar plate to provide the one or more colonies of CaCO₃-producing bacteria of step (a).

With preference, step (a) comprises providing a single colony of CaCO₃-producing bacteria and step vi comprises extracting a single colony from said agar plate to provide the single colony of CaCO₃-producing bacteria of step (a).

For example, the CaCO₃-producing bacteria are one or more urease-producing bacteria. With preference, said one or more urease-producing bacteria are one or more bacteria selected from Vibrio strain, Bacillus strain, Lysinibacillus strain, Sporosarcina strain, Kocuria strain, Halomonas strain and/or Pseudomonas strain; more preferably from Vibrio strain.

For example, the one or more bacteria selected from Bacillus strain are or comprise one or more of Bacillus sp. CR2, B. pasteurii NCIM 2477, B. megateriumSS3, or B. thuringiensis.

For example, a bacterium selected from Lysinibacillus strain is Lysinibacillus sphaericus CH5.

For example, a bacterium selected from Sporosarcina strain is Sporosarcina pasteurii.

For example, a bacterium selected from Kocuria strain is K. flava CR1.

For example, a bacterium selected from Halomonas strain is Halomonas sp. SR4.

For example, the one or more bacteria selected from Vibrio strain are or comprise Vibrio crassostrae, Vibrio rotiferianus, Vibrio jasicida 090810c, Vibrio coralliilyticus, Vibrio cyclitrophicus, Vibrio mediterranei, Vibrio lentus, Vibrio splenditus, Vibrio kanaloae, Vibrio coralliirubri, Vibrio natriegens NBRC 15636, Vibrio nigripulchritudo POn4, Vibrio parahaemolyticus, Vibrio furnissii, Vibrio tubiashii; more preferably Vibrio crassostrae.

For example, the first marine broth and the second marine broth each comprise seawater and one or more nutrients.

For example, the first marine broth and the second marine broth are identical or different.

Whichever embodiment is selected, step (d) of incubating is preferably carried out under stirring settings. With preference, said stirring settings comprise stirring said dissolved inoculated culture and/or said supernatant cell culture at a stirring speed ranging between 70 rpm and 130 rpm.

Alternatively, whichever embodiment is selected, step (d) of incubating is preferably carried out under static settings.

With preference, and whichever embodiment is selected, one or more of the following features can be used to further define step (d):
the incubating conditions of step (d) comprise an incubating temperature ranging between 15° C. and 30° C.
step (d) of incubating is carried out under aerobic settings.

Advantageously, the second marine broth comprises between 2.7 g/l and less than 4.0 g/l of CaCl₂ and the mixture of biogenic crystals of calcium carbonate formed at step (d) comprises calcite and aragonite in a weight ratio of calcite over aragonite that is superior to 1, preferentially determined by Raman spectroscopy analysis and/or by microscopy imaging (i.e., scanning electron microscopy) since calcite is an elongated polymorph and aragonite is a spherical polymorph. For example, the second marine broth comprises at least 2.5 g/l of CaCl₂, more preferably at least 2.6 g/l of CaCl₂, even more preferably at least 2.7 g/l of CaCl₂, most preferably at least 3.0 g/l of CaCl₂. This allows obtaining a mixture with a weight ratio of calcite/aragonite superior to 1. For example, the second marine broth comprises between at least 2.5 g/l of CaCl₂ and at most 3.9 g/l of CaCl₂, preferably between at least 2.6 g/l of CaCl₂ and at most 3.7 g/l of CaCl₂,

Advantageously, the second marine broth comprises between 4.0 g/l and 6.5 g/l of CaCl₂ and the mixture of biogenic crystals of calcium carbonate formed at step (d) comprises calcite and is aragonite-free. For example, the second marine broth comprises at least 4.0 g/l of CaCl₂, more preferably at least 4.5 g/l of CaCl₂, even more preferably at least 5.0 g/l of CaCl₂, most preferably at least 5.5 g/l of CaCl₂ or even most preferably at least 6.0 g/l of CaCl₂. This allows obtaining only calcite, and therefore triple or even quadruple the yield of calcite. For example, the second marine broth comprises between at least 4.0 g/l of CaCl₂ and at most 6.5 g/l of CaCl₂, preferably between at least 4.1 g/l of CaCl₂ and at most 6.4 g/l of CaCl₂, more preferably between at least 4.2 g/l of CaCl₂ and at most 6.3 g/l of CaCl₂.

For example, the second marine broth comprises between 2.5 g/l and 6.5 g/l of CaCl₂, or between 2.6 g/l and 6.3 g/l of CaCl₂, or between 2.7 g/l and 6.0 g/l of CaCl₂, or between 2.8 g/l and 5.5 g/l of CaCl₂, or between 2.7 g/l and 6.5 g/l of CaCl₂.

In an embodiment, the second marine broth advantageously comprises one or more cationic surfactants at a concentration ranging between 0.001 mM and 0.01 mM. For example, the one or more cationic surfactants are selected from quaternary ammonium salts, such as one or more cationic surfactants selected from cetytrimethylammonium bromide (CTAB), cetylpyridinium chloride (CPC), benzalkonium chloride (BAC), benzethonium chloride (BZT), dimethyldioctadecylammonium chloride or dioctadecyldimethylammonium bromide (DODAB). With preference, the second marine broth comprises cetytrimethylammonium bromide (CTAB). This allows for increasing the density of the calcium carbonate crystals that are recovered.

In another embodiment, the second marine broth advantageously comprises one or more surfactants selected from anionic surfactants, cationic surfactants and non-ionic surfactants. For example, the second marine broth comprises one or more surfactants at a concentration of at least 0.05 nM, preferably of at least 0.1 nM.

. For example, an anionic surfactant is sodium dodecyl sulfate (SDS), a cationic surfactant is cetytrimethylammonium bromide (CTAB) and a non-ionic surfactant is polysorbate 20. This allows for aggregating the calcium carbonate crystals that are recovered.

For example, the inoculating conditions of step (b) comprise one or more of the following features:
a temperature ranging between 15° C. and 30° C.; and/or
an inoculating period of at least 24 hours; and/or
an inoculation under static settings; and/or
an inoculation under aerobic settings.

In an embodiment step (e) is carried out and comprises the following sub-steps:
i. washing with water said mixture of biogenic crystals of calcium carbonate;
ii. optionally, drying said mixture of biogenic crystals of calcium carbonate with air.

### DESCRIPTION OF FIGURES

FIG. 1: Raman spectrum showing the signature of aragonite (no additional CaCl₂ is present in the second marine broth).

FIG. 2: Raman spectrum showing the signature of calcite (0.9 g/l of CaCl₂ have been added into the second marine broth).

FIG. 3: Raman spectrum showing the signature of calcite (1.8 g/l of CaCl₂ have been added into the second marine broth).

FIG. 4: Raman spectrum showing the signature of calcite (2.7 g/l of CaCl₂ have been added into the second marine broth).

FIG. 5: Raman spectrum showing the signature of calcite (3.6 g/l of CaCl₂ have been added into the second marine broth).

FIG. 6: Raman spectrum showing the signature of calcite (4.5 g/l of CaCl₂ have been added into the second marine broth).

FIG. 7: Scanning electron microscopy (SEM) image of aragonite (obtained in the absence of additional CaCl₂ in the second marine broth).

FIG. 8: SEM image of calcite (obtained when additional 0.9 g/l of CaCl₂ have been added into the second marine broth).

FIG. 9: Aspect ratio of crystals of calcium carbonate (CCC) upon different conditions.

FIG. 10: Mean diameter of CCC measured by SEM upon different conditions.

FIG. 11: Volume of individual CCC (in μm³) upon different conditions.

FIG. 12: Density of CCC produced (in μm³ of CCC produced per μm² of production area).

FIG. 13: Bright field images of CCC (i.e., aragonite) (obtained when 9 mM of SDS have been added into the second marine broth).

FIG. 14: Bright field images of CCC (i.e., aragonite) (obtained when 0.9 mM of SDS have been added into the second marine broth).

FIG. 15: Bright field zoomed-in images of CCC (i.e., aragonite) (obtained when 0.9 mM of SDS have been added into the second marine broth).

FIG. 16: Bright field images of CCC (i.e., aragonite) (obtained when 0.09 mM of SDS have been added into the second marine broth).

FIG. 17: Bright field zoomed-in images of CCC (i.e., aragonite) (obtained when 0.09 mM of SDS have been added into the second marine broth).

FIG. 18: Bright field images of CCC (i.e., aragonite) (obtained when 0.1 mM of CTAB have been added into the second marine broth).

FIG. 19: Aspect ratio of CCC upon different conditions (via the supernatant embodiment).

FIG. 20: Mean diameter of the crystals of calcium carbonate (CCC) measured by SEM under different conditions (via the supernatant embodiment).

FIG. 21: Volume of individual CCC (in μm³) upon different conditions (via the supernatant embodiment).

FIG. 22: Density of CCC produced (in μm³ of CCC produced per μm² of production area) (via the supernatant embodiment).

FIG. 23: Image of the formation of the seeds when stirring occurs during the incubation step.

FIG. 24: Bright field microscopy image of growing CCC from CCC seeds

FIG. 25: Fluorescent microscopy image of growing CCC from CCC seeds.

FIG. 26: Bright field microscopy images of CCC that are placed into dodecane. The images on the left are images taken at day zero of the stability experiment, the images in the centre are images taken on day 2 of the stability experiment and the images on the right are images taken on day 5 of the stability experiment. All the images have identical scale bars, as the one depicted on the image at the bottom right corner.

FIG. 27: Bright field microscopy images of CCC that are placed into deionized water (MilliQ® water). The images on the left are images taken at day zero of the stability experiment, the images in the centre are images taken on day 2 of the stability experiment and the images on the right are images taken on day 5 of the stability experiment. All the images have identical scale bars, as the one depicted on the image at the bottom right corner.

FIG. 28: Bright field microscopy images of CCC that are placed into artificial seawater, i.e., water comprising sea salt at a concentration of 31 g/l. The images on the left are images taken at day zero of the stability experiment, the images in the centre are images taken on day 2 of the stability experiment and the images on the right are images taken on day 5 of the stability experiment. All the images have identical scale bars, as the one depicted on the image at the bottom right corner

### DETAILED DESCRIPTION

For the disclosure, the following definitions are given:

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The recitation of numerical ranges by endpoints includes all integer numbers and, where appropriate, fractions subsumed within that range (e.g., 1 to 5 can include 1, 2, 3, 4, 5 when referring to, for example, a number of elements, and can also include 1.5, 2, 2.75 and 3.80, when referring to, for example, measurements). The recitation of endpoints also includes the recited endpoint values themselves (e.g., from 1.0 to 5.0 includes both 1.0 and 5.0). Any numerical range recited herein is intended to include all sub-ranges subsumed therein.

The expression “marine broth” refers to a growth medium which has a composition that mimics seawater and thus helps marine bacteria to grow abundantly. For example, a marine broth contains the nutrients, salts and trace elements which are required for the growth of marine bacteria.

The step of “ageing a culture” consists in letting said culture into its suspension during a certain period of time, without acting on its environment.

The expression “static setting” in chemistry and/or biochemistry refers to operating conditions that are performed without stirring and/or without shaking.

The particular features, structures, characteristics or embodiments may be combined in any suitable manner, as would be apparent to a person skilled in the art from this disclosure, in one or more embodiments.

The present disclosure involves the production of one or more crystals of calcium carbonate in the presence of a marine bacterium that is not pathogenic to humans. This bacteria is isolated from the marine environment without known prior gene modification. The technique used here can be seamlessly extended to other microorganism populations or communities thereof, under carefully controlled environmental factors, including temperature, pH, presence of particulate impurities, aerobic/anaerobic conditions, salinity, fluid flow, light conditions, and local gas concentrations.

In particular, the present disclosure relates to a process for producing biogenic crystals of calcium carbonate, said process is remarkable in that it comprises the following steps:
a) providing one or more colonies of CaCO₃-producing bacteria;
b) inoculating said one or more colonies in a first marine broth under inoculating conditions to form an inoculated culture;
c) adding a second marine broth to the inoculated culture to form a dissolved inoculated culture and/or collecting a supernatant of the inoculated culture and adding a second marine broth to said supernatant;
d) incubating said dissolved inoculated culture and/or said supernatant cell culture under incubating conditions to form a mixture of biogenic crystals of calcium carbonate,
e) optionally, recovering said mixture of biogenic crystals of calcium carbonate.

With preference, step (a) comprises providing a single colony of CaCO₃-producing bacteria.

More particularly, the disclosure provides a process for producing biogenic crystals of calcium carbonate remarkable in that it comprises the following steps:
a) providing one or more colonies of CaCO₃-producing bacteria;
b) inoculating said one or more colonies in a first marine broth under inoculating conditions to form an inoculated culture;
c) adding a second marine broth to the inoculated culture to form a dissolved inoculated culture and/or ageing the inoculated culture to form an aged inoculated culture, centrifuging said aged inoculated culture to obtain a supernatant, collecting the supernatant, and adding a second marine broth to said supernatant to form a dissolved supernatant,
   wherein the second marine broth is selected to comprise less than 2.0 g/l of CaCl₂ or at least 2.5 g/l of CaCl₂;
d) incubating said dissolved inoculated culture and/or said dissolved supernatant under incubating conditions to form a mixture of biogenic crystals of calcium carbonate,
e) optionally, recovering said mixture of biogenic crystals of calcium carbonate.

Step (c) corresponds in other terms to a step of preparing from the inoculated culture formed at step (b) a dissolved inoculated culture in a second marine broth; and/or of preparing a mixture of supernatant of the inoculated culture formed at step (b) and a second marine broth to form a supernatant cell culture.

For example, when the second marine broth is selected to comprise less than 2.0 g/l of CaCl₂, the second marine broth comprises 1.9 g/l of CaCl₂ or less, preferably 1.8 g/l of CaCl₂ or less, or 1.7 g/l of CaCl₂ or less, or 1.6 g/l of CaCl₂ or less, or 1.5 g/l of CaCl₂ or less. In that case, only aragonite is obtained.

For example, when the second marine broth is selected to comprise at least 2.5 g/l of CaCl₂, the second marine broth comprises between 2.6 g/l of CaCl₂ and less than 4.0 g/l of CaCl₂, or between 2.7 g/l of CaCl₂ and less than 4.0 g/l of CaCl₂, or between 2.8 g/l of CaCl₂ and 3.9 g/l of CaCl₂. In that case, biogenic crystals of calcium carbonate with a weight ratio of calcite over aragonite that is superior to 1 are obtained.

For example, when the second marine broth is selected to comprise at least 2.5 g/l of CaCl₂, the second marine broth comprises between at least 4.0 g/l of CaCl₂ and 6.5 g/l of CaCl₂ or more, or between 4.1 g/l of CaCl₂ and 6.4 g/l of CaCl₂, or between 4.2 g/l of CaCl₂ and 6.3 g/l of CaCl₂, or between 4.3 g/l of CaCl₂ and 6.2 g/l of CaCl₂, or between 4.2 g/l of CaCl₂ and 6.1 g/l of CaCl₂. In that case, only calcite is obtained.

To prepare the one or more colonies of CaCO₃-producing bacteria, CaCO₃-producing bacteria, such as one or more urease-producing bacteria, are first provided.

With preference, said one or more urease-producing bacteria are one or more bacteria selected from Vibrio strain, Bacillus strain, Lysinibacillus strain, Sporosarcina strain, Kocuria strain, Halomonas strain and/or Pseudomonas strain; more preferably from Vibrio strain. Other urease-producing bacteria can be selected from Proteus strain, Morganella strain, Serratia strain, Clostridium strain, Fusobacterium strain, Ureaplasma strain, Providencia strain, Sarcina strain, Lactobacillus strain, Streptococcus strain, and Enterobacter strain.

For example, the one or more bacteria selected from Bacillus strain are or comprise one or more of Bacillus sp. CR2, B. pasteurii NCIM 2477, B. megateriumSS3, or B. thuringiensis.

For example, a bacterium selected from Lysinibacillus strain is Lysinibacillus sphaericus CH5.

For example, a bacterium selected from Sporosarcina strain is Sporosarcina pasteurii.

For example, a bacterium selected from Kocuria strain is K. flava CR1.

For example, a bacterium selected from Halomonas strain is Halomonas sp. SR4.

For example, the one or more bacteria selected from Vibrio strain are or comprise Vibrio crassostrae, Vibrio rotiferianus, Vibrio jasicida 090810c, Vibrio coralliilyticus, Vibrio cyclitrophicus, Vibrio mediterranei, Vibrio lentus, Vibrio splenditus, Vibrio kanaloae, Vibrio coralliirubri, Vibrio natriegens NBRC 15636, Vibrio nigripulchritudo POn4, Vibrio parahaemolyticus, Vibrio furnissii, Vibrio tubiashii; more preferably Vibrio crassostrae.

If they are provided as a cell culture on a solid agar substrate, as is generally the case, the cell culture is then removed (using, for example, a cell culture loop) and then immersed into a culture medium for example a marine broth. This is necessary to maintain the CaCO₃-producing bacteria alive since such bacteria are heterotrophic bacteria, meaning that they cannot produce their own food. For example, marine broth comprises seawater and one or more nutrients. To this aspect, seawater is water comprising for example sea salt at a concentration ranging between 25 g/l and 35 g/l.

The one or more nutrients of the first and/or second marine broth can advantageously be selected from peptone (for example at a concentration ranging between 3 g/l and 7 g/l), yeast extract (for example at a concentration ranging between 0.8 g/l and 1.2 g/l), sodium chloride (for example at a concentration ranging between 18 g/l and 22 g/l), magnesium chloride (for example at a concentration ranging between 5.5 g/l and 6.5 g/l), calcium chloride (for example at a concentration ranging between 1.5 g/l and 2.5 g/l), boric acid (for example at a concentration ranging between 20 mg/l and 24 mg/l).

For example, the first and/or second marine broth comprises between 2.5 g/l and 6.5 g/l of CaCl₂, more preferably between 2.6 g/l and 6.3 g/l of CaCl₂, even more preferably between 2.7 g/l and 6.0 g/l of CaCl₂, or most preferably between 2.8 g/l and 5.5 g/l of CaCl₂, or between 2.7 g/l and 6.5 g/l of CaCl₂.

Advantageously, to obtain a mixture having a weight ratio between calcite and aragonite superior to 1, the second marine broth comprises at least 2.5 g/l of CaCl₂, more preferably at least 2.6 g/l of CaCl₂, even more preferably at least 2.7 g/l of CaCl₂, most preferably at least 3.0 g/l of CaCl₂. For example, the second marine broth comprises between at least 2.5 g/l of CaCl₂ and at most 3.9 g/l of CaCl₂, preferably between at least 2.6 g/l of CaCl₂ and at most 3.7 g/l of CaCl₂. The weight ratio is preferentially determined by Raman spectroscopy analysis and/or by microscopy imaging since calcite is an elongated polymorph and aragonite is a spherical polymorph. In particular, microscopy imaging, such as scanning electron microscopy, allows for determining the aspect ratio. To this aspect, aragonite has an aspect ratio ranging between 0.80 and 0.90 (and thus rather spherical), while calcite has an aspect ratio ranging between 0.60 and 0.70 (and thus less spherical in comparison to the aragonite samples).

Advantageously, to obtain only calcite, and subsequently increase the yield of calcite, the second marine broth comprises at least 4.0 g/l of CaCl₂, more preferably at least 4.5 g/l of CaCl₂, even more preferably at least 5.0 g/l of CaCl₂, most preferably at least 5.5 g/l of CaCl₂ or even most preferably at least 6.0 g/l of CaCl₂. For example, the second marine broth comprises between at least 4.0 g/l of CaCl₂ and at most 6.5 g/l of CaCl₂, preferably between at least 4.1 g/l of CaCl₂ and at most 6.4 g/l of CaCl₂, more preferably between at least 4.2 g/l of CaCl₂ and at most 6.3 g/l of CaCl₂.

To prepare the second marine broth with the required amount of CaCl₂ in it for either producing a mixture of calcite and aragonite with a predominant amount of calcite or producing only calcite, an example of commercially available marine broth is Difco™ marine broth 2216. Such commercially available marine broth already comprises 1.8 g/l of CaCl₂. To add CaCl₂, the Difco™ marine broth 2216 broth is mixed in boiling water for 1 min, followed by a standard autoclave procedure (e.g., 121° C. for 15 minutes) to make the medium sterile. Then, additional CaCl₂ is added to the autoclaved broth and sonicated for 5 min.

The addition of glycerol allows for freezing the culture medium comprising the CaCO₃-producing bacteria. For example, the freezing temperature can be comprised between −70° C. and −90° C., or between −75° C. and −85° C. The freezing step preserves the cells and their biological properties.

Then, the frozen stock of bacteria is streaked on an agar plate (for example by using a cell culture loop). The streaking is a technique to isolate one or more colonies (such as a single colony) of bacteria from a sample, using a selective culture medium. The culture medium used for the streaking is a marine broth comprising agar. The culture medium used for the streaking can be prepared as follows. A marine broth comprising agar can be mixed with a marine broth devoid of agar to form the selective culture medium. For example, the marine broth with agar can be in a concentration ranging between 45 g/l and 65 g/l, more preferably between 50 g/l and 60 g/l. For example, the marine broth devoid of agar can be in a concentration ranging between 30 g/l and 45 g/l, more preferably between 35 g/l and 40 g/l. Alternatively, only a marine broth with agar can be used, still at a concentration ranging between 45 g/l and 65 g/l, more preferably between 50 g/l and 60 g/l. The aqueous solution comprising the two marine broths, or only the marine broth comprising agar, is then mixed thoroughly and then heated under stirring up to boiling until the powder is completely dissolved, followed by a standard autoclave procedure (e.g., 121° C. for 15 minutes) to make the medium sterile.

The one or more nutrients of the marine broth comprising agar can advantageously be selected from peptone (for example at a concentration ranging between 3 g/l and 7 g/l), yeast extract (for example at a concentration ranging between 0.8 g/l and 1.2 g/l), sodium chloride (for example at a concentration ranging between 18 g/l and 22 g/l), magnesium chloride (for example at a concentration ranging between 5.5 g/l and 6.5 g/l), calcium chloride (for example at a concentration ranging between 1.5 g/l and 2.5 g/l), boric acid (for example at a concentration ranging between 20 mg/l and 24 mg/l) and agar (for example at a concentration ranging between 10 g/l and 20 g/l).

An example of commercially available marine broth with agar is Difco™ marine agar 2216, which comprises a concentration of agar of 15 g/l. An example of commercially available marine broth devoid of agar is Difco™ marine broth 2216.

The streaked plates can be then incubated for at least 12 hours before the extraction of one or more colonies of CaCO₃-producing bacteria.

The inoculating step (b) consists in placing the one or more colonies of CaCO₃-producing bacteria into a medium that contains the essential nutrients for growth. This creates a cell culture, in which the CaCO₃-producing bacteria is put into the best conditions for accomplishing its purpose. Upon incubation, it is possible thanks to the components of the medium, to influence how the CaCO₃-producing bacteria will work to form the biogenic crystals of calcium carbonate such as calcite and/or aragonite. Said biogenic crystals can thus be called microbial biogenic tunable structures (μ-BITS) since it is indeed possible to modify the composition of the marine broth. The formation of such μ-BITS provides a highly competitive and sustainable alternative to plastic-based materials, as well as for industries that rely on filler materials/minerals, mined often under extremely hazardous and compromised environmental and safety standards.

The inoculating step (b) is advantageously carried under smooth conditions, meaning that it can be performed at room temperature (i.e., at a temperature ranging between 15° C. and 30° C., or between 18° C. and 25° C.) and under aerobic settings which are less cumbersome to establish in comparison to the anaerobic settings. Also, the inoculating step (b) is carried out for at least 24 hours and/or under static settings.

In a first embodiment, the incubation step (d) is performed on the dissolved inoculated culture. The incubation step (d) is performed within the second marine broth, that has been used to dissolve the inoculated culture. With preference, the ratio between the inoculated culture and the second marine broth is ranging between 1/200 and 1/20, preferably; between 1/180 and 1/50; more preferably, between 1/150 and 1/100. As the remaining bacteria or the cell debris have not been removed, when the optional step (e) of recovering said calcite and/or said aragonite is carried out, it can be necessary to microfilter the one or more crystals to remove the contaminants.

In a second embodiment, the issue related to the unwanted debris or crystals of calcium carbonate formed during step (b) can be avoided since the inoculated culture is centrifuged to obtain a supernatant which will be collected and a precipitate which can be discarded. It is noted that by simply mixing the supernatant with fresh media, the culture still grows and precipitated crystals can be observed since it is not possible to remove completely the bacteria from centrifugation. Also, before the centrifugation, the inoculated culture is aged, for example during a period ranging between 2 days and 7 days, more preferably during a period ranging between 5 days and 7 days. The ageing can among others increase the density of the CCC productions due to the reduction of the volume of the fresh media over time. With preference, when the supernatant is collected, the ratio between the supernatant and the second marine broth during the incubation step (d) is ranging between 1/3 and 3/1; preferably, between 1/2 and 2/1. By collecting the supernatant and achieving the formation of the calcite and/or the aragonite starting from the supernatant, it is also shown that the polydispersity of said calcite and/or said aragonite is also enhanced. In addition, the use of supernatant also reduces the volume of the second marine broth required for the production of CCC, for a given volume of cell culture. This reduction of volume can reduce the cost of production of CCC by lowering the amount of marine broth required for CCC production.

Whichever embodiment is selected, step (d) of incubating can be carried out under static settings. However, when said step (d) of incubating is carried out under stirring settings, in the absence of additional CaCl₂ into the second marine broth during the incubation step, these particular conditions involving stirring and/or shaking favour the formation of non-spherical aragonite, which is an exotic morphology of this usually spherical polymorph. One could also employ both static and stirring/shaking culture conditions, depending on the desired particle size. With preference, said stirring settings comprise stirring said dissolved inoculated culture and/or said supernatant cell culture at a speed ranging between 70 rpm and 130 rpm, preferably between 80 rpm and 120 rpm, or between 90 rpm and 110 rpm. These particular conditions involving stirring and/or shaking can also break down the already-formed crystals, which will thereafter coalesce together to form a sheet of the biogenic crystals of calcium carbonate such as calcite and/or aragonite.

In the present process, the incubating step (d) can be carried out at room temperature, namely at a temperature ranging between 15° C. and 30° C., or between 18° C. and 25° C. The incubating step (d) can also be carried out in aerobic settings.

Advantageously, the second marine broth comprises at least 2.7 g/l of CaCl₂ (for example, the Difco™ marine broth 2216 with at least 0.9 g/l of CaCl₂ in addition), more preferably at least 3.6 g/l of CaCl₂ (for example, the Difco™ marine broth 2216 with at least 1.8 g/l of CaCl₂ in addition). This allows obtaining a mixture of biogenic crystals of calcium carbonate comprising calcite and aragonite with a weight ratio of calcite over aragonite that is superior to 1 and this is the first example of how to adapt the process to the desired need.

In addition, when the second marine broth comprises advantageously at least 4.5 g/l of CaCl₂ (for example, the Difco™ marine broth 2216 with at least 2.7 g/l of CaCl₂ in addition), more preferably at least 5.4 g/l of CaCl₂ (for example, the Difco™ marine broth 2216 with at least 3.6 g/l of CaCl₂ in addition) or even more preferably at least 6.3 g/l of CaCl₂ (for example, the Difco™ marine broth 2216 with at least 4.5 g/l of CaCl₂ in addition) it has been demonstrated that the mixture of biogenic crystals of calcium carbonate is aragonite-free and that it allows obtaining only calcite and subsequently generate a high yield of said particular polymorph. The process of the present disclosure is therefore not only advantageous in selecting the type of polymorphs but also can provide, at least for calcite, a high yield of it.

In a second example of how to adapt the process to the desired need, the second marine broth can comprise one or more cationic surfactants at a concentration ranging between 0.001 mM and 0.01 mM, preferably at a concentration ranging between 0.002 mM and 0.009 mM, or between 0003 mM and 0.008 mM. These particular conditions allow for increasing the density of the calcium carbonate crystals that are recovered.

However, in the case the second marine broth would be loaded with one or more anionic surfactants and/or one or more cationic surfactants and/or one or more non-ionic surfactants, the loading of said one or more surfactants amounting preferably to a concentration of at least 0.05 nM, or at least 0.10 nm, or at least 0.1 mM, or of at least 0.2 mM, this allows for aggregating the calcium carbonate crystals that are recovered.

It is noted that the addition of the one or more cationic surfactants at a concentration ranging between 0.001 mM and 0.01 mM into the second marine broth can also occur concomitantly with the presence of at least 2.7 g/l of CaCl₂ in the second marine broth (for example, the Difco™ marine broth 2216 with at least 0.9 g/l of CaCl₂ in addition), to increase the density of the calcite that are recovered. If no CaCl₂ is added, then, it is the density of aragonite that is increased. Similarly, the addition of the one or more anionic surfactants and/or one or more cationic surfactants at a concentration of at least 0.1 mM concomitantly with the presence of at least 2.7 g/l of CaCl₂ into the second marine broth (for example, the Difco™ marine broth 2216 with at least 0.9 g/l of CaCl₂ in addition) is used for increasing the aggregation of the calcite crystals and rendering their size smaller. If the concentration of CaCl₂ in the second marine broth is less than 2.7 g/l, preferably less than 2.6 g/l, more preferably less than 2.5 g/l, even more preferably less than 2.4 g/l, then the aggregation of aragonite is increased, along with its decrease in size.

For example, the one or more cationic surfactants are selected from quaternary ammonium salts. In particular, the one or more cationic surfactants are selected from cetytrimethylammonium bromide (CTAB), cetylpyridinium chloride (CPC), benzalkonium chloride (BAC), benzethonium chloride (BZT), dimethyldioctadecylammonium chloride or dioctadecyldimethylammonium bromide (DODAB); more preferably, an example of cationic surfactant is cetytrimethylammonium bromide (CTAB).

For example, the one or more anionic surfactants are selected from alkylbenzene sulfonates, alkyl sulfonates, alkyl sulfonates, alkyl sulfates, salts of fluorinated fatty acids, silicones, fatty alcohol sulfates, polyoxyethylene fatty alcohol ether sulfates, α-olefin sulfonate, polyoxyethylene fatty alcohol phosphates ether, alkyl alcohol amide, alkyl sulfonic acid acetamide, alkyl succinate sulfonate salts, amino alcohol alkylbenzene sulfonates, naphthenates, alkylphenol sulfonate and polyoxyethylene monolaurate. In particular, an example of an anionic surfactant is sodium dodecyl sulfate (SDS).

For example, the one or more non-ionic surfactants are selected from polysorbate 20 (Tween® 20), Triton® X series, the Span series, the Pluronic® series, Brij™. In particular, an example of non-ionic surfactant is polysorbate 20.

The incubation step (d) can last at least 3 days. Indeed, the crystals are us usually seen around 3 or 4 days after the start of the incubation. It is advantageous to wait at least 7 days to carry out step (e) when it is desired to recover the biogenic crystals of calcium carbonate such as calcite and/or aragonite. The crystals are mainly found on the water/air interface, although some products are also found at the base of the well surface. The crystals are washed and then they can be dried. In the case of the incubation step (d) being performed on the dissolved inoculated culture, it is recommended to use a microfilter to separate the crystals from the remaining bacteria and/or the cell debris. Once the crystals have been recovered, they are washed with water, preferably with MilliQ® water. It is preferred that the crystals are washed several times with water, preferably with MilliQ® water, for example, they are washed at least 5 times. Then, by using for example a micropipette, the crystals are removed and they can be dried in air.

### Test and Determination Methods

### Raman Spectroscopy

The Raman measurements were performed with a Renishaw inVia micro-Raman spectrometer. The excitation laser has a wavelength of 785 nm and output power of around 100 mW focused on the sample through a Leica 50X long-distance objective with numerical aperture NA=0.5. The spectrum is analysed with a 1200 gr/mm grating resulting in a spectra resolution of ˜1.3 cm⁻¹. Acquisition time was 120 s unless stated otherwise.

### Scanning Electron Microscopy (SEM)

SEM is performed using Jeol JSM-6010 in SEI mode, 20 kV, Working distance 10-12 mm and spot size=50. The samples were first fixed on an SEM imaging stub and coated with 5-10 nm gold before the SEM imaging.

### Determination of the Volume of Individual CCC

Fluorescence images of the CCC were processed in MATLAB to tabulate the 2D-area of the CCC produced. Afterwards, by assuming CCC produced to be spherical, the diameter of each analysed CCC is tabulated by the formula diameter=2×(Area/4π)^{1/2}. Finally, based on the diameter obtained from this approximation, the volume of each CCC is calculated from the formula volume=(4/3)π(diameter/2)³.

### Determination of the Aspect Ratio of the CCC

The aspect ratio corresponds to the ratio between the minimum Feret diameter and the maximum Feret diameter, the Feret diameter being the measure of an object's size along a specified direction. The aspect ratio is obtained thanks to the measurements made by scanning electron microscopy.

### Determination of the Density of the CCC Production

Fluorescence images were processed in MATLAB to tabulate the diameter of the CCC produced. Afterwards, by assuming CCC produced to be approximately spherical, the total volume of CCC analysed is tabulated based on the diameters obtained in the image analysis. Finally, the volume of each CCC is summed up, and divided by the area of image analysed. Hence, this gives rise to the volume of CCC/area of image analysed, and this quotient here is used to estimate the density of the CCC production.

### Bright-Field Microscopy

Bright-field imaging is performed using Carl Zeiss Imager D1 upright microscope, and the images are captured using a 5X objective (N.A.=0.16) and microscope camera (FLIR, Model: GS3-U3-41C6M-C) that was adapted to a 0.5X camera adaptor. The formation of CaCl₂ crystals is largely formed at the cell culture/air-fluid interface.

### Polarizing Microscopy

Polarizing images are performed on a Nikon polarized light microscope (Eclipse LV100N-POL, Nikon) using 5X, 10X, 20X, or 40X objectives. In some instances, a 530 nm phase-retardation plate during the image acquisition.

### Fluorescence Microscopy

Fluorescence imaging is performed using Carl Zeiss Imager D1 upright microscope, and the images are captured using a 5X objective (N.A.=0.16) and microscope camera (FLIR, Model: GS3-U3-41C6M-C) that was adapted to a 0.5X camera adaptor. In addition, fluorescence optical filter cubes (FITC/GFP excitation/emission of 470 nm/525 nm and Cy3 excitation/emission 550 nm/605 nm) were used to obtain the respective fluorescence images.

The formation of CaCl₂ crystals is largely formed at the cell culture/air-fluid interface.

### EXAMPLES

The embodiments of the present disclosure will be better understood by looking at the experimental details below.

### Preparation of Marine Broth Without Agar

The liquid media, which is free of agar, is prepared by dissolving Difco™ marine broth 2216 to a concentration of 37.4 g/L. In detail, 400 ml of MilliQ® water was added to a 500-ml blue cap bottle, and the bottle is placed in a water bath with a temperature set to 102-104° C. such that the water is noticeably boiling. Meanwhile, a magnetic stirrer is added to the bottle. Then, after the water is boiling, 14.96 g of Difco™ marine broth 2216 is then added to the bottle with the stirrer set to 600-1000 rpm. The stirring continues for 1 min after the Difco™ marine broth 2216 is added to the bottle. After this, the bottle is removed from the water bath and autoclaved.

### Preparation of Marine Broth with Agar

400 ml of MilliQ® water was added to a 500-ml blue cap bottle, and the bottle is placed in a water bath with a temperature set to 102-104° C. such that the water is noticeably boiling. Meanwhile, a magnetic stirrer is added to the bottle. Then, after the water is boiling, 22.04 g of Difco™ marine agar 2216 is then added to the bottle with the stirrer set to 600-1000 rpm. The stirring continues for 1 min after the Difco™ marine agar 2216 is added to the bottle. After this, the bottle is removed from the water bath and autoclaved. After the autoclave cycle, the agar solution is poured into a 10-cm diameter sterile, polystyrene Petri dish to have a continuous layer of agar solution (˜15 ml), which is done so within a biosafety cabinet (BSC). The lids of the Petri dishes are left ajar, and the Petri dishes are left to cool down for a couple of hours or overnight in the BSC. After cooling, the Petri dishes are covered, inverted (hardened agar is on top of the petri dish cover), and sealed with Parafilm®. Then, these agar plates are stored in a 2-8° C. fridge before use.

### Preparation of the Single Colony of CaCO₃-Producing Bacteria

DSM 17220 Vibrio crassostrae (also designated with strain named LGP-7) is purchased from Leibniz Institute DSMZ-German Collection of Microorganisms and Cell Cultures GmbH as a live culture, which was received as a culture on a solid agar substrate. The agar culture is removed using a cell culture loop and immersed into a marine broth devoid of agar, for a culture of around 23 h. Over here, 4 ml of marine broth devoid of agar was added to a 10-ml cell culture tube. This liquid cell culture is mixed 1:1 in volume with 50% (w/w) glycerol, by adding 500 μl of liquid cell culture to 500 μl of 50% (w/w) glycerol, and this is done within a cryogenic plastic vial. This final mixture is stored in a −80° C. freezer.

The frozen stock is streaked on an agar plate using a cell culture loop and grown overnight in an incubator at 24° C. The agar solution here is prepared in the same manner as the Difco™ 2216 cell media (which is the marine broth devoid of agar), but, as explained above, Difco™ 2216 marine broth is replaced by Difco™ marine agar 2216, with a concentration of 55.1 g/L.

From the overnight streaked plates, one single colony on the agar culture is extracted by cell culture loops.

### Biogenic Formation of One or More Crystals of CaCO₃

The single colony, mixed with a first marine broth, i.e., 10 ml of Difco™ marine broth 2216, is incubated at 24° C. under static conditions for 1 day to 15 days. This liquid culture was performed in a 50-ml Erlenmeyer flask under aerobic settings. An inoculated culture is thus formed.

### First Embodiment: Via the Dissolved Inoculated Culture

The inoculating culture is then dissolved into a second marine broth, i.e., Difco™ marine broth 2216, within a 6-well plate, which is a polystyrene well plate with untreated well surfaces. In detail, 8 ml of Difco™ marine broth 2216 is added to each well, and 80 μl of the inoculated culture is added to each well, and the final cell culture mixture is mixed by pipetting up and down the cell culture mixture for 5 to 10 times.

The well plates are then incubated at room temperature (about 20° C.) conditions, for example in static conditions to obtain calcium carbonate crystals in the form of aragonite and/or calcite. For the interstitial spaces within the well plates that do not contain the cell culture mixture, 4 ml of MilliQ® water was added.

The products are seen usually around 3-4 days of incubation. They can be observed under optical microscopy under bright field, phase contrast, dark field, and fluorescence modes with wide-ranging optical filter setups, not limited to FITC and Cy3 excitation/emission filters. The products are produced in the range of 10-150 μm after 7 days, and they are mainly found on the water/air interface, although some products were found at the base of the well surface.

The products, namely the crystals of aragonite, are recoverable from 7 days onwards after the beginning of the incubation, but they are still recoverable after more than this period. To recover the products, the most of the cell culture is removed from the well. Then, MilliQ® water is added to wash the products. The used MilliQ® water is then removed. Fresh MilliQ® water is added again, and this process is repeated 8 times in total. Finally, the products are removed using a micropipette and dispensed on a glass slide. In another method, after washing the products with MilliQ® water as described above, a glass cover slip (18 mm×18 mm×0.15 mm) is used to immerse near the water/air interface and removed, such that the products are wetted on the glass cover slip to form a thin layer of products. In both cases, the products are dried in the air.

The dried products are analysed using polarizing microscopy, optical microscopy, fluorescence microscopy, scanning electron microscopy, and Raman spectroscopy.

### Production of Calcite or of Aragonite

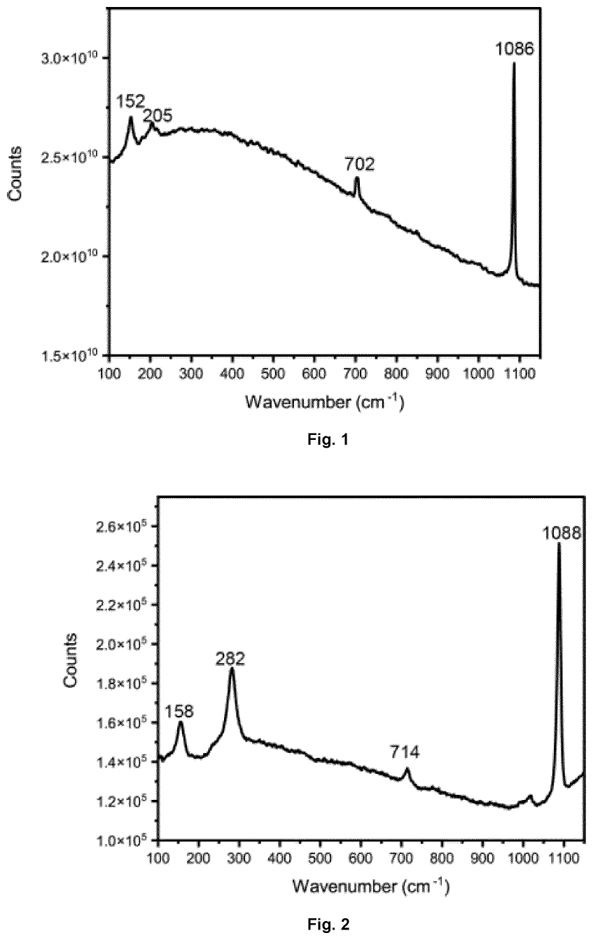

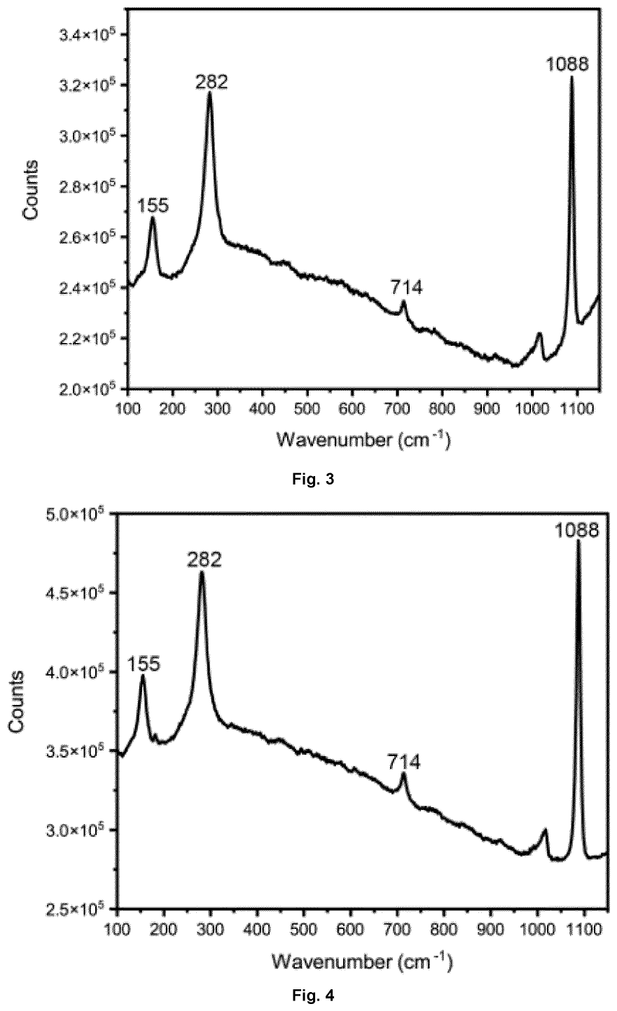

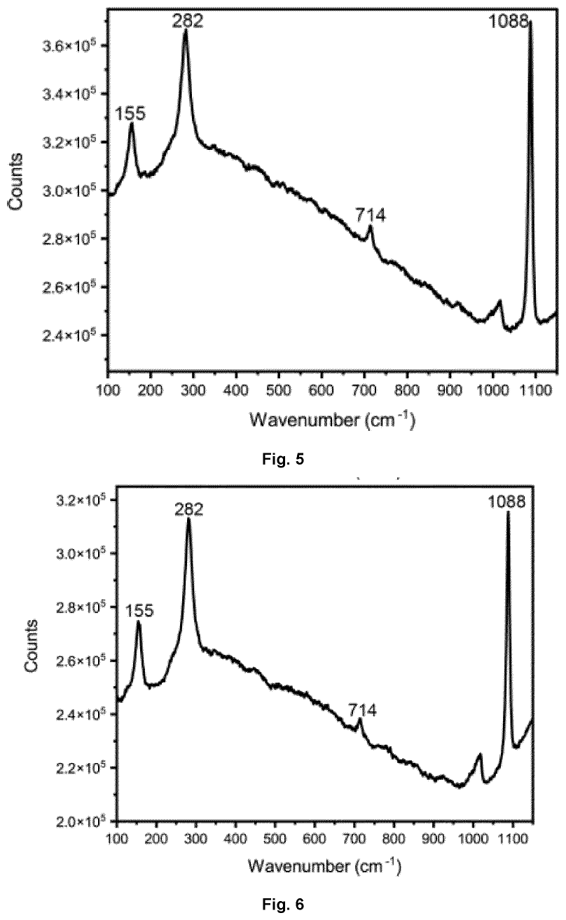

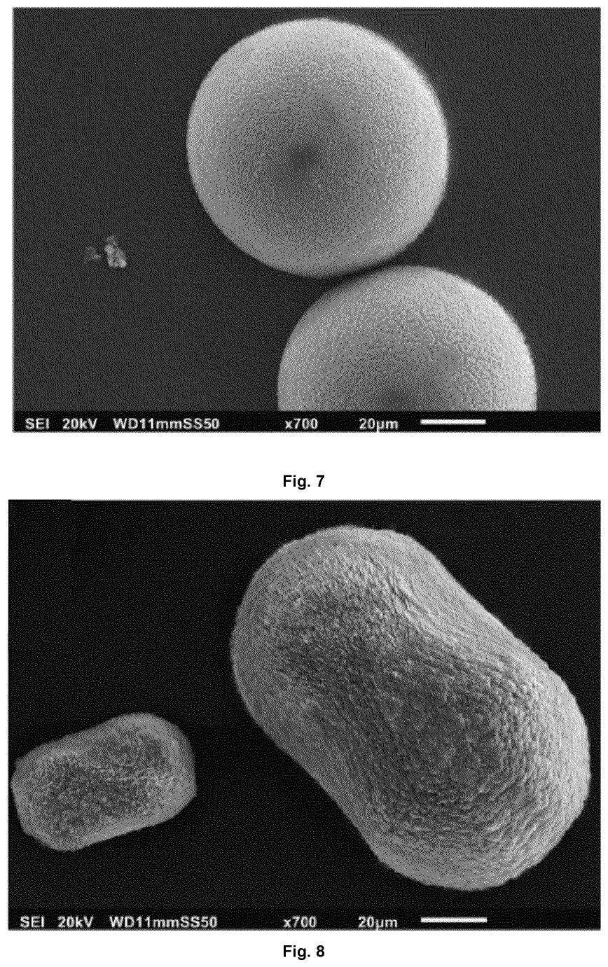

By introducing additional calcium chloride into the second marine broth, namely into Difco™ marine broth 2216, a mixture of calcium carbonate with a predominant amount of calcite over aragonite has been obtained. FIG. 1 shows indeed the formation of aragonite in the absence of additional CaCl₂ (peak at 205 cm⁻¹) while FIGS. 2 to 6 show the formation of calcite when the second marine broth presents an additional concentration of CaCl₂ amounting to 0.9 g/l, 1.8 g/l, 2.7 g/l, 3.6 g/l, and 4.5 g/l, the peak having shifted to 282 cm⁻¹, indicating the signature of calcite. FIG. 7 is the SEM images of the morphology of the calcium carbonate crystals obtained when no CaCl₂ has been added into the second marine broth while FIG. 8 shows the SEM images of calcite obtained upon addition in the second marine broth of CaCl₂ in a concentration of 0.9 g/l.

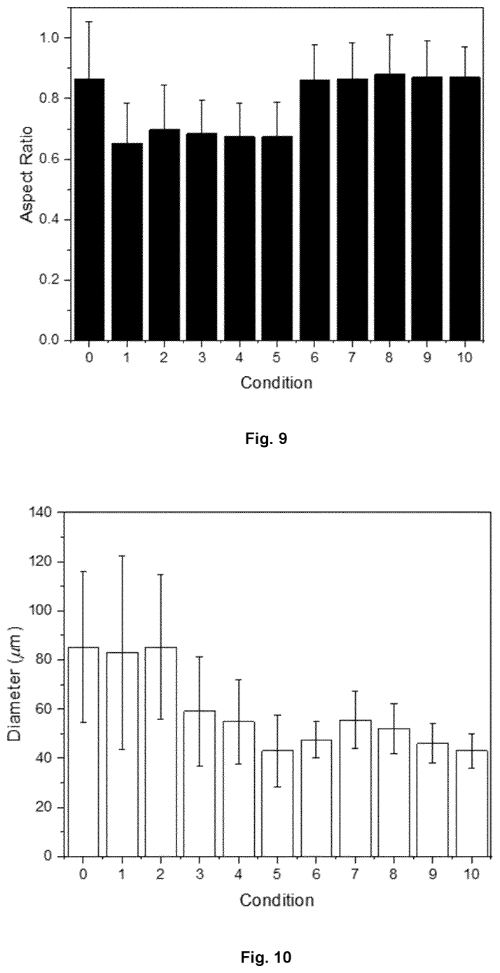

FIGS. 9 to 12 respectively show the aspect ratio, the mean diameter, the volume, and the density of the CaCO₃ crystals (CCC) under the following conditions:
-0-: no additional CaCl₂ is present in the second marine broth so the total concentration of CaCl₂ in said second marine broth being of 1.8 g/l of CaCl₂
-1-: 0.9 g/l of CaCl₂ has been added to the second marine broth so that the total concentration of CaCl₂ in the second marine broth (which is Difco™ marine broth 2216) is 2.7 g/l of CaCl₂
-2-: 1.8 g/l of CaCl₂ have been added to the second marine broth so that the total concentration of CaCl₂ in the second marine broth (which is Difco™ marine broth 2216) is 3.6 g/l of CaCl₂
-3-: 2.7 g/l of CaCl₂ have been added to the second marine broth so that the total concentration of CaCl₂ in the second marine broth (which is Difco™ marine broth 2216) is 4.5 g/l of CaCl₂
-4-: 3.6 g/l of CaCl₂ have been added to the second marine broth so that the total concentration of CaCl₂ in the second marine broth (which is Difco™ marine broth 2216) is 5.4 g/l of CaCl₂
-5-: 4.5 g/l of CaCl₂ have been added to the second marine broth so that the total concentration of CaCl₂ in the second marine broth (which is Difco™ marine broth 2216) is 6.3 g/l of CaCl₂
-6-: no additional CaCl₂ is present in the second marine broth while 0.01 mM of CTAB has been added in the second marine broth
-7-: no additional CaCl₂ is present in the second marine broth while 0.001 mM of CTAB has been added in the second marine broth
-8-: no additional CaCl₂ is present in the second marine broth while 5 nM of Tween® 20 has been added in the second marine broth
-9-: no additional CaCl₂ is present in the second marine broth while 0.5 nM of Tween® 20 has been added in the second marine broth
-10-: no additional CaCl₂ is present in the second marine broth while 0.05 nM of Tween® 20 has been added in the second marine broth

FIG. 9, which represents the aspect ratio of the CCC that was obtained, also shows (see conditions -0-,-6-,-7-,-8-,-9-, and -10-) that in the absence of additional CaCl₂, the aspect ratio is close to 1 (between 0.80 and 0.90), confirming the sphericity of the aragonite shown on FIG. 7. For the calcite, the aspect ratio is between 0.60 and 0.70.

FIG. 10 shows that the diameter of aragonite can be ranging between 40 μm and 90 μm, as determined by SEM measurements. The addition of CTAB and Tween® 20 into the second marine broth allows for decreasing the diameter of the aragonite, the diameter ranging then between 30 μm and 60 μm.

FIG. 10 also shows that the diameter of the calcite crystals is ranging between 40 μm and 90 μm, as determined by SEM measurements. The diameter of the calcite crystals is decreasing upon the addition of CaCl₂ into the second marine broth. It is noted that although calcite is not spherical but rather elongated, the term “diameter” used when referring to calcite crystals is based on an assumption that calcite is spherical only for determining its size based on the area determined by SEM measurements.

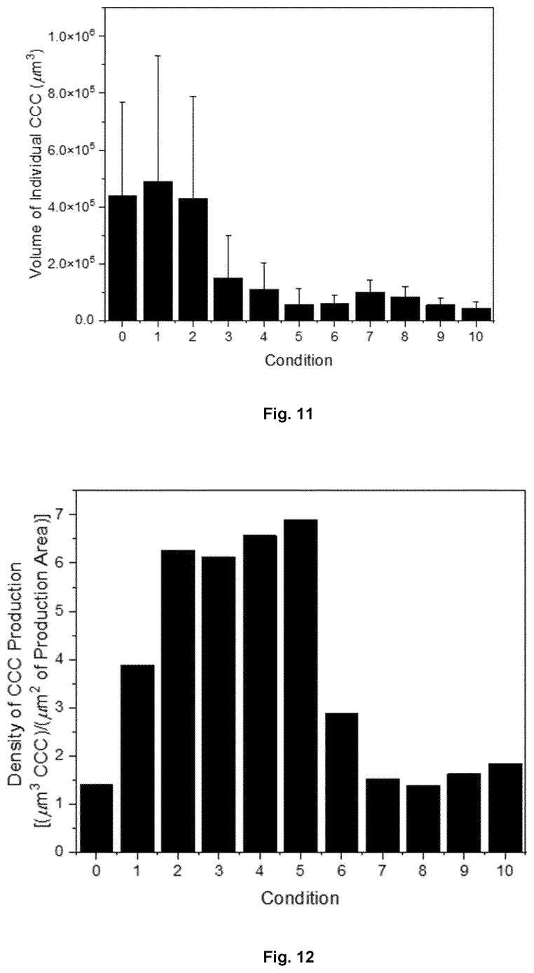

FIG. 11 indicates the volume of the CCC. It has been shown that upon addition of CaCl₂, the volume of calcite decreases (from about 5×10⁵ μm³ (condition -1-) to about 0.5×10⁵ μm³ (condition -5-) and that upon addition of CTAB, the volume of aragonite is lower than without addition of CTAB (about 4×10⁵ μm³—condition -0-). However, when a low concentration of CTAB (0.001 mM) is added to the second marine broth (condition -7-), the volume of the aragonite (about 1.0×10⁵ μm³) is higher than when a higher concentration of CTAB (0.01 mM) is added (condition -6-, indicating a volume of about 0.6×10⁵ μm³). When a low concentration of Tween® 20 (0.05 nM) is added to the second marine broth (condition -10-), the volume of the aragonite (about 0.4×10⁵ μm³) is lower than when higher concentrations of Tween® 20 (0.5 nM and 5 nM) are added (condition -9-, indicating a volume of about 0.6×10⁵ μm³, and condition -8- indicating a volume of about 0.8×10⁵ μm³).

As shown in FIG. 12, the addition of 0.01 mM CTAB (conditions -6-) allows for increasing the density of the aragonite that is produced. By comparison with the condition -0-, wherein the density of aragonite production was below 1.5 μm³ per μm² of production area, the addition of CTAB at a concentration of 0.01 mM allows for obtaining a density of aragonite at a value of 2.9 μm³ per μm² of production area. The addition of a lower concentration of CTAB into the second marine broth (see condition -7-) also allows the increase of the density, although to a lesser extent (1.5 μm³ per μm² of production area).

The addition of 0.5 nM and 0.05 nM of Tween® 20 allows for increasing the density of the aragonite that is produced. By comparison with the condition -0-, wherein the density of aragonite production was below 1.5 μm³ per μm² of production area, the addition of Tween® 20 at a concentration of 0.5 nM allows for obtaining a density of aragonite at a slightly higher concentration of 1.6 μm³ per μm² of production area. For a concentration of 0.05 nM Tween® 20, it allows for obtaining a density of aragonite at a higher concentration of 1.8 μm³ per μm² of production area.

FIG. 12 also indicates that the incorporation of at least 1.8 g/l of CaCl₂ into the second marine broth (condition -2-) allows the obtaining of CCC (i.e., calcite) at a density superior to 6 μm³ per μm² of production area, while in the absence of CaCl₂ (condition -0-), the obtaining of CCC (i.e., aragonite) was inferior to 1.5 μm³ per μm² of production area. Upon the addition of a bigger amount of CaCl₂ into the second marine broth (condition -5-), the density of CCC (i.e., calcite) has even reached 6.9 μm³ per μm² of production area.

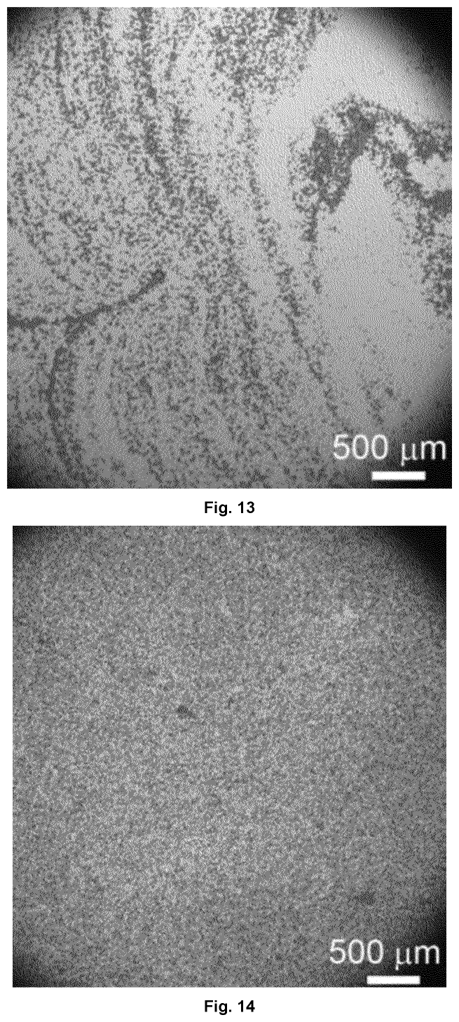

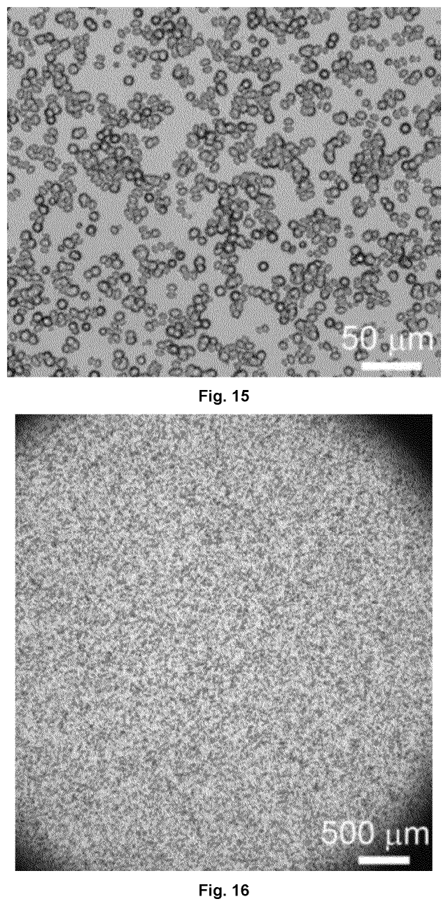

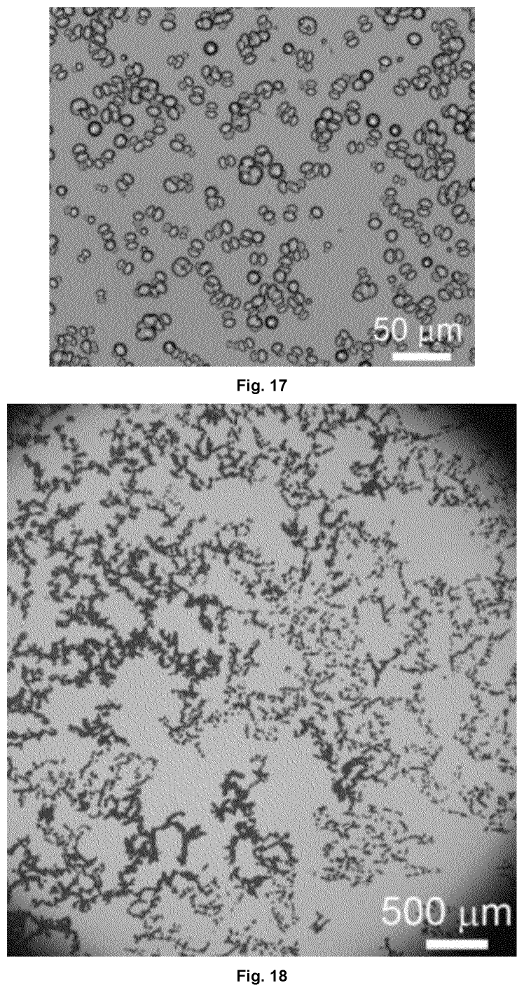

Experiments consisting of the addition of sodium dodecyl suffete (SDS) into the second marine broth at various concentration (respectively 9 mM, 0.9 mM, and 0.09 mM) results in the increase of the aggregation of the calcium carbonate crystals and the decrease of the size of the crystals (when no additional CaCl₂ is added to the second marine broth being Difco™ marine broth 2216, namely when the total concentration of CaCl₂ is of 1.8 g/l). This is shown on FIGS. 13 to 17. Zoomed-in images on FIGS. 15 and 17 show dumbbell-shaped aragonite. FIG. 18 shows similar trends (i.e., increase of aggregation and decrease of the size of the aragonite crystals) but when 0.1 mM of CTAB has been added into the second marine broth.

### Second Embodiment: Via the Collection of the Supernatant

First, from streaked agar plates, a single colony is picked and introduced into a first cell media broth and grown for around 24 hours. Then, this overnight cell culture is diluted in a second cell media broth in a typical ratio of 1:100 within 6-well plates.

This second inoculated/diluted culture is aged for 2 days up to 7 days. Then, it is removed from the wells and added to 2-ml centrifuge tubes. After which, the aged inoculated cultures are centrifuged for 10 min at 4° C., at 1500 G (4000 rpm). The supernatant of the centrifuged cell culture is removed and added to the well pates, and different ratios of the supernatant to a second marine broth can be used, ranging from 0% supernatant to 100% supernatant. The supernatant is called the supernatant cell cultures. Total volumes in each well are kept as 8 ml.

The well plates are then incubated in room temperature conditions, in static conditions. For the interstitial spaces within the well plates that do not contain the cell culture mixture, 4 ml of MilliQ® water was added.

The products are seen usually around 3-4 days of incubation. They can be observed under optical microscopy under bright field, phase contrast, dark field, and fluorescence modes with wide-ranging optical filter setups, not limited to FITC and Cy3 excitation/emission filters. The products are produced in the range of 10-100 μm after 7 days, and they are mainly found on the water/air interface, although some products were found at the base of the well surface.

The products are recoverable from 7 days onwards after the beginning of the incubation, but they are still recoverable after more than this period. To recover the products, they are removed using a micropipette and/or using a cover glass/slip and dried in air.

The dried products are analysed using polarizing microscopy, optical microscopy, fluorescence microscopy, scanning electron microscopy, and Raman spectroscopy.

### Production of Aragonite

By ageing the inoculated culture, centrifuging it and collecting the supernatant, it is possible to obtain crystals of calcium carbonate. This has allowed enhancing the polydispersity of the CCC.

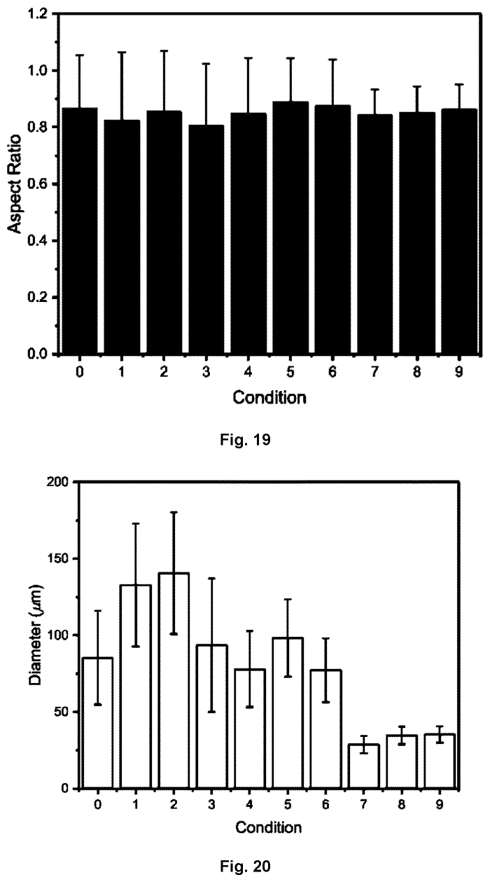

FIGS. 19 to 22 respectively show the aspect ratio, the mean diameter, the volume, and the density of the CaCO3 crystals (CCC) under the following conditions:
-0-: no additional CaCl₂ is present in the second marine broth so the total concentration of CaCl₂ in said second marine broth being of 1.8 g/l of CaCl₂.
and no supernatant has been added. The initial culture was aged for 15 days, before diluting it 1:100 into the second marine broth.
-1-: the inoculated culture has been aged for 2 days and the ratio between the supernatant and the second marine broth is 3:1.
-2-: the inoculated culture has been aged for 2 days and the ratio between the supernatant and the second marine broth is 1:1.
-3-: the inoculated culture has been aged for 2 days and the ratio between the supernatant and the second marine broth is 1:3.
-4-: the inoculated culture has been aged for 5 days and the ratio between the supernatant and the second marine broth is 3:1.
-5-: the inoculated culture has been aged for 5 days and the ratio between the supernatant and the second marine broth is 1:1.
-6-: the inoculated culture has been aged for 5 days and the ratio between the supernatant and the second marine broth is 1:3.
-7-: the inoculated culture has been aged for 7 days and the ratio between the supernatant and the second marine broth is 3:1.
-8-: the inoculated culture has been aged for 7 days and the ratio between the supernatant and the second marine broth is 1:1.
-9-: the inoculated culture has been aged for 7 days and the ratio between the supernatant and the second marine broth is 1:3.

FIG. 19, which represents the aspect ratio of the CCC that was obtained, shows that the aspect ratio is close to 1 (between 0.80 and 0.90), indicating that the crystals are of spherical shape. This is an indication that aragonite crystals are formed under all 10 conditions when performing the incubation from the supernatant.

FIG. 20 shows that the diameter of aragonite can be ranging between 25 μm and 150 μm, as determined by SEM measurements. A smaller size can be obtained upon ageing the inoculated culture.

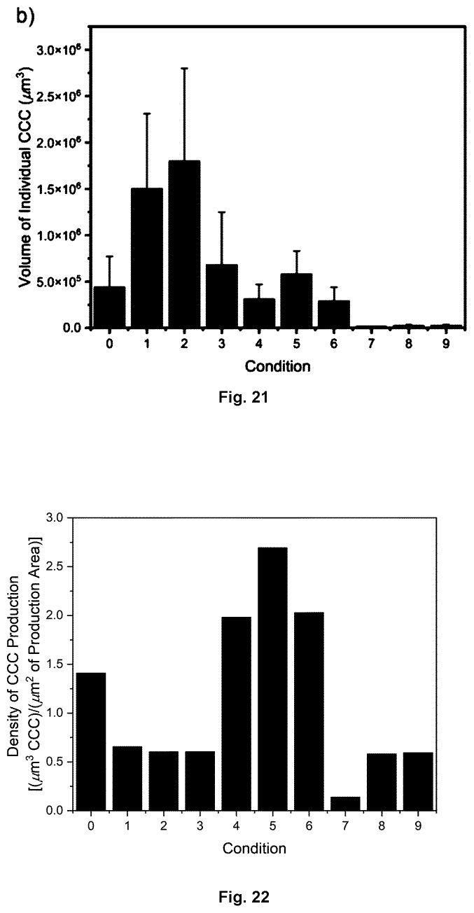

FIG. 21 indicates the volume of the CCC. The measurement of the volume confirms the findings shown in FIG. 20, that upon ageing, the size of the crystals decreases.

FIG. 22 indicates that upon ageing for 5 days in the inoculated culture, the density of the aragonite crystals is ranging between 1.8 mm³ per μm² of production area and 2.8 mm³ per μm² of production area.

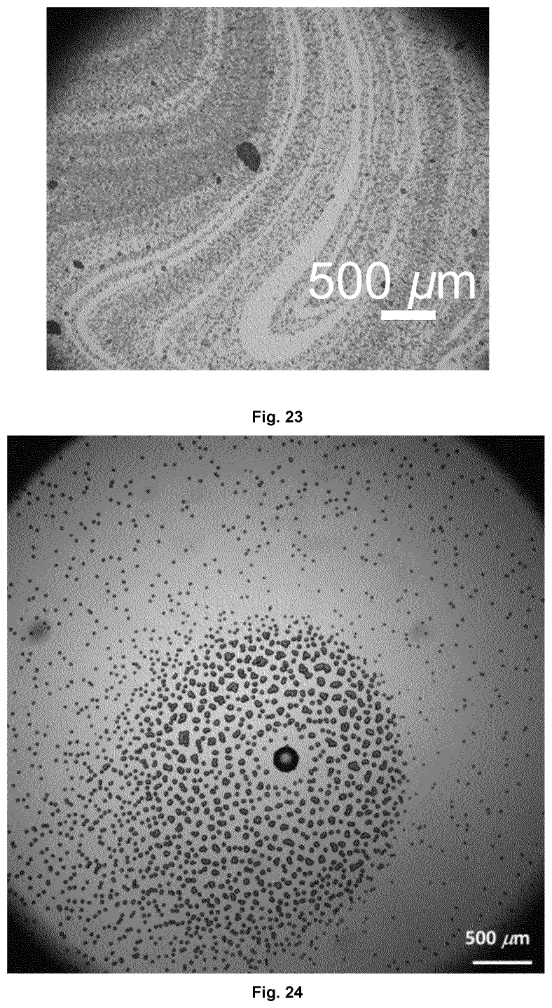

During the incubation of the supernatant, when stirring is provided, the formation of non-spherical aragonite is triggered. Indeed, stirring breaks down the particles into smaller sizes. Stirring was achieved at 100 rpm. The smaller size particles, resulting from the stirring during incubation, also act as seeding points for subsequent growth of CCC. FIG. 23 is a micrograph obtained by SEM/Bright field microscopy showing the generation of minuscule structures, each of which being able to act as independent seeding sites.

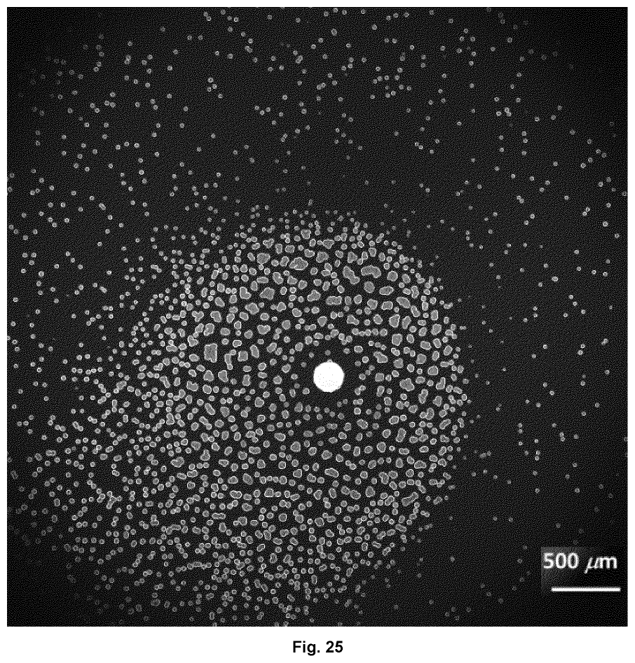

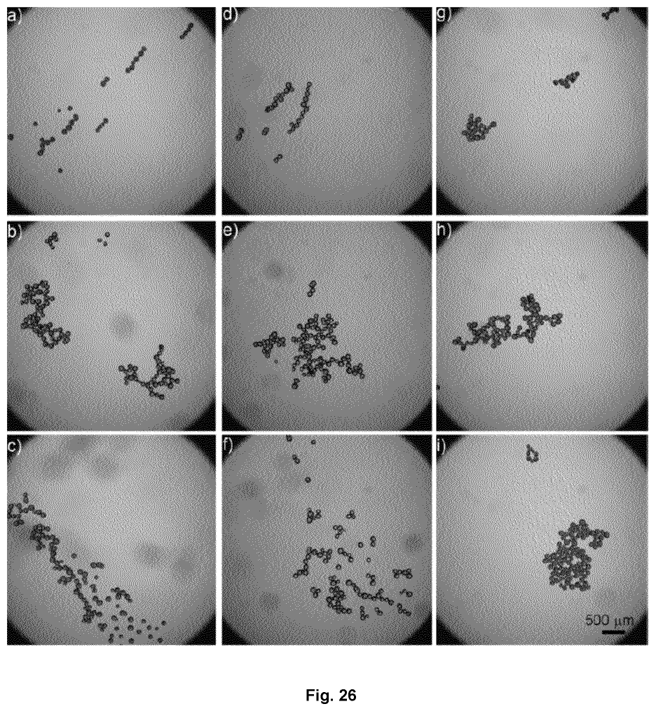

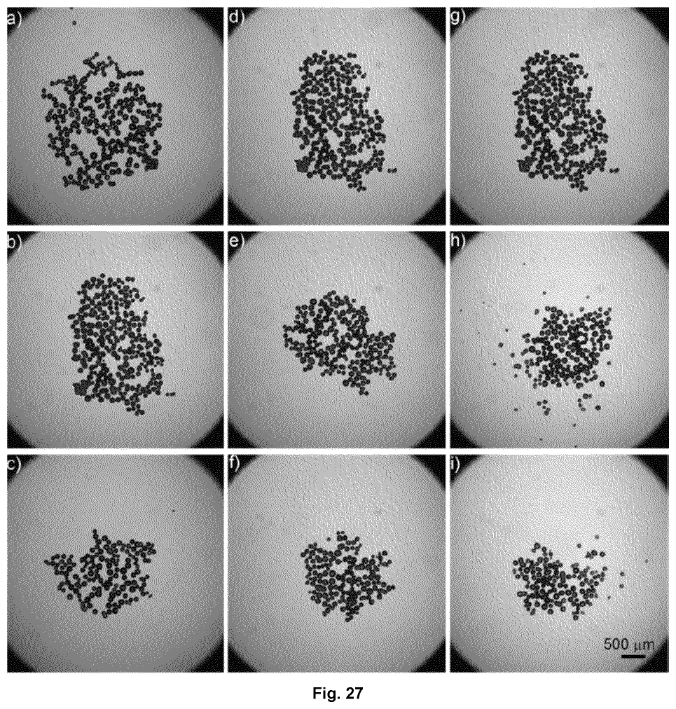

FIGS. 24 and 25 are respectively a bright-field microscopy image and a fluorescence microscopy image of the growing aragonite from CCC seeds. It can be seen that due to the stirring, the broken-down particles tend to coalesce together, forming sheets of aragonite. Then, if there is no additional CaCl₂ in the medium, aragonite becomes non-spherical aragonite and sheets of non-spherical aragonite can be formed. In contrast, when there is additional CaCl₂ in the medium (i.e., at least 2.5 g/l or preferably at least 2.7 g/l), calcite is formed and then sheets of calcite can be generated

### Stability Experiments of the Crystals of Calcium Carbonate

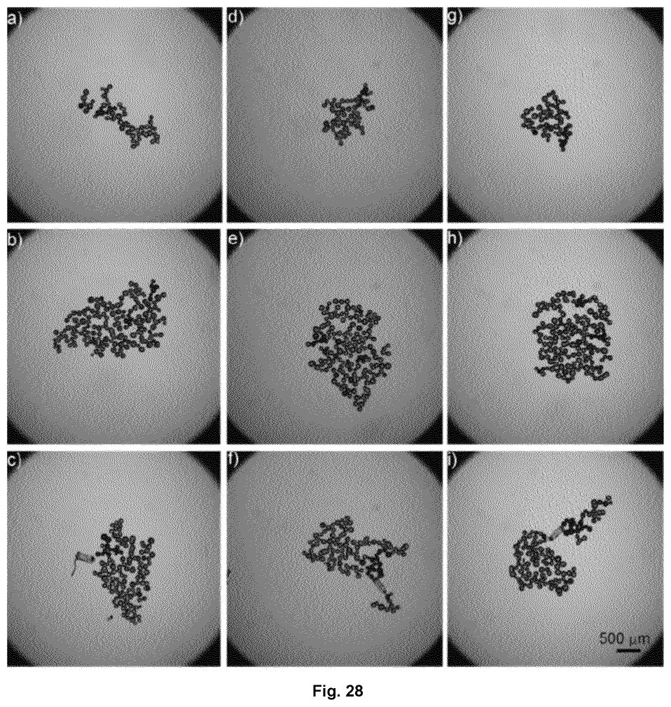

Stability experiments were conducted on the recovered crystals of calcium carbonate. FIGS. 26, 27 and 28 respectively show those stability experiments conducted into dodecane, deionized water (i.e., MilliQ® water) and artificial seawater (i.e., water comprising sea salt at a concentration of 31 g/l). It can be seen that the crystals remain stable, showing no signs of physical degradation throughout the experiments.

1. A process for producing biogenic crystals of calcium carbonate characterized in that it comprises the following steps:
a) providing one or more colonies of CaCO₃-producing bacteria;
b) inoculating said one or more colonies in a first marine broth under inoculating conditions to form an inoculated culture;
c) adding a second marine broth to the inoculated culture to form a dissolved inoculated culture and/or ageing the inoculated culture to form an aged inoculated culture, centrifuging said aged inoculated culture to obtain a supernatant, collecting the supernatant, and adding a second marine broth to said supernatant to form a dissolved supernatant,
wherein the second marine broth is selected to comprise less than 2.0 g/l of CaCl₂ or at least 2.5 g/l of CaCl₂;
d) incubating said dissolved inoculated culture and/or said dissolved supernatant under incubating conditions to form a mixture of biogenic crystals of calcium carbonate,
e) optionally, recovering said mixture of biogenic crystals of calcium carbonate.

2. The process according to claim 1 is characterized in that when the second marine broth is selected to comprise less than 2.0 g/l of CaCl₂, the second marine broth comprises 1.8 g/l of CaCl₂ or less.

3. (canceled)

4. (canceled)

5. The process according to claim 1 characterized in that the second marine broth comprises one or more cationic surfactants at a concentration ranging between 0.001 mM and 0.01 mM.

6. The process according to claim 5 is characterized in that the one or more cationic surfactants are selected from quaternary ammonium salts.

7. The process according to claim 5 is characterized in that the one or more cationic surfactants are selected from cetytrimethylammonium bromide (CTAB), cetylpyridinium chloride (CPC), benzalkonium chloride (BAC), benzethonium chloride (BZT), dimethyldioctadecylammonium chloride or dioctadecyldimethylammonium bromide (DODAB).

8. (canceled)

9. The process according to claim 1 is characterized in that the second marine broth comprises one or more surfactants selected from anionic surfactants, cationic surfactants and non-ionic surfactants.

10. The process according to claim 9 is characterized in that the second marine broth comprises one or more surfactants at a concentration of at least 0.05 nM.

11. The process according to claim 1 is characterized in that, in the dissolved inoculated culture, the ratio between the inoculated culture and the second marine broth is ranging between 1/200 and 1/20.

12. (canceled)

13. The process according to claim 1 is characterized in that the step (d) of incubating is carried out under stirring settings.

14. (canceled)

15. The process according to claim 1 is characterized in that step (d) of incubating is carried out under static settings.

16. (canceled)

17. The process according to claim 1 is characterized in that the step (d) of incubating is carried out under aerobic settings.

18. The process according to claim 1 is characterized in that the CaCO₃-producing bacteria are one or more urease-producing bacteria.

19. (canceled)

20. The process according to claim 1 is characterized in that the first marine broth and the second marine broth each comprise seawater and one or more nutrients.

21. The process according to claim 1 is characterized in that the process comprises the following steps to achieve step (a):
i. providing a culture medium with living CaCO₃-producing bacteria, said culture medium being a marine broth;
ii. adding glycerol in said culture medium;
iii. freezing said culture medium to obtain a frozen stock;
iv. streaking said frozen stock on an agar plate;
V. incubating the agar plate for at least 12 hours;
vi. extracting one or more colonies from said agar plate to provide the one or more colonies of CaCO₃-producing bacteria of step (a).

22. The process according to claim 1 is characterized in that the inoculating conditions of step (b) comprise a temperature ranging between 15° C. and 30° C.

23. The process according to claim 1 is characterized in that the inoculating step (b) is carried out for a period of at least 24 hours.

24. The process according to claim 1 is characterized in that the inoculating step (b) is carried out under static settings.

25. The process according to claim 1 is characterized in that the inoculating step (b) is caried out under aerobic settings.

26. The process according to claim 1 is characterized in that step (e) is carried out and in that said step (e) comprises the sub-step of washing with water said mixture of biogenic crystals of calcium carbonate.

27. The process according to claim 26 is characterized in that step (e) further comprises the sub-step of drying said mixture of biogenic crystals of calcium carbonate with air.
